(19) United States
(12) Patent Application Publication
(10) Pub. No.: US 2024/0169162 A1
(43) Pub. Date: May 23, 2024

(54) LANGUAGE PROCESSING APPARATUS, LANGUAGE PROCESSING METHOD, AND PROGRAM

(71) Applicants: NEC CORPORATION, Tokyo (JP); NATIONAL UNIVERSITY CORPORATION NARA INSTITUTE OF SCIENCE AND TECHNOLOGY, Ikoma-shi (JP)

(72) Inventors: Yutaka UNO, Tokyo (JP); Aki ANDO, Nara (JP); Shuntaro YADA, Nara (JP); Shoko WAKAMIYA, Nara (JP); Eiji ARAMAKI, Nara (JP)

(73) Assignees: NEC CORPORATION, Tokyo (JP); NATIONAL UNIVERSITY CORPORATION NARA INSTITUTE OF SCIENCE AND TECHNOLOGY, Ikoma-shi (JP)

(21) Appl. No.: 18/514,564

(22) Filed: Nov. 20, 2023

(30) Foreign Application Priority Data
Nov. 22, 2022 (JP) 2022-186502

(51) Int. Cl. G06F 40/40; G16H 15/00

(52) U.S. Cl. CPC G06F 40/40; G16H 15/00

(57) ABSTRACT

A language processing apparatus, a language processing method, and a program capable of assisting a user or the like in accounting for the result of language processing are provided. A language processing apparatus includes a unique expression extraction unit configured to extract a unique expression related to medical care from text information, and a language processing unit configured to perform language processing related to medical care based on the unique expression.

### INCORPORATION BY REFERENCE

This application is based upon and claims the benefit of priority from Japanese patent application No. 2022-186502, filed on Nov. 22, 2022, the disclosure of which is incorporated herein in its entirety by reference.

### TECHNICAL FIELD

The present disclosure relates to a language processing apparatus, a language processing method, and a program.

### BACKGROUND ART

A technology for assisting a user or the like in preparing a medical document has been proposed. In Patent Literature 1 (Liu, P. J.: Learning to Write Notes in Electronic Health Records, arXiv preprint arXiv: 1808.02622 2018), generating a template, automatically interpolating text or the like, and correcting typographic errors by using a language model are attempted. Patent Literature 2 (Jeblee, S., Khan Khattak, F., Crampton, N., Mamdani, M., and Rudzicz, F.: Extracting relevant information from physician-patient dialogues for automated clinical note taking, in Proceedings of the Tenth International Workshop on Health Text Mining and Information Analysis (LOUHI 2019), pp. 65-74, Hong Kong (2019), Association for Computational Linguistics) discloses a technology for extracting eight types of expressions unique to medical care including names of symptoms and generating a SOAP (Subjective Objective Assessment Plan)-type medical record using these expressions. Patent Literature 3 (Enarvi, S., Amoia, M., Del-Agua Teba, M., Delaney, B., Diehl, F., Gallopyn, G., Hahn, S., Harris, K., McGrath, L., Pan, Y., Pinto, J., Rubini, L., Ruiz, M., Singh, G., Stemmer, F., Sun, W., Vozila, P., Lin, T., and Ramamurthy, R.: Generating Medical Reports from Patient-Doctor Conversations Using Sequence-to-Sequence Models, in Proceedings of the First Workshop on Natural Language Processing for Medical Conversations, pp. 22-30, Online 2020, Association for Computational Linguistics) discloses a technology for generating a draft of a SOAP (Subjective Objective Assessment Plan)-type medical record from text of a clinical conversation in an end-to-end manner without recognizing entities.

When end-to-end language processing is performed, there is a problem that the result cannot be easily interpreted and it is difficult to account therefor (i.e., to explain the problem) which is required in medical institutions.

The present disclosure has been made to solve the above-described problem, and an object thereof is to provide a language processing apparatus, a language processing method, and a program capable of assisting a user or the like in accounting for the result of language processing.

### SUMMARY

A language processing apparatus according to the present disclosure includes:

a unique expression extraction unit configured to extract a unique expression related to medical care from text information; and

a language processing unit configured to perform language processing related to medical care based on the unique expression.

A language processing method according to the present disclosure includes:

extracting, by a computer, a unique expression related to medical care from text information; and

performing, by the computer, language processing related to medical care based on the unique expression.

A program according to the present disclosure causes a computer to perform:

a process for extracting a unique expression related to medical care from text information; and

a process for performing language processing related to medical care based on the unique expression.

### BRIEF DESCRIPTION OF DRAWINGS

The above and other aspects, features and advantages of the present disclosure will become more apparent from the following description of certain example embodiments when taken in conjunction with the accompanying drawings, in which:

FIG. 1 is a block diagram showing a configuration of a language processing apparatus according to a first example embodiment;

FIG. 2 is a block diagram showing a configuration of a language processing apparatus according to a second example embodiment;

FIG. 3 is a table for explaining event data according to the second example embodiment; and

FIG. 4 is a table for explaining documents generated in the second example embodiment.

### EXAMPLE EMBODIMENT

### First Example Embodiment

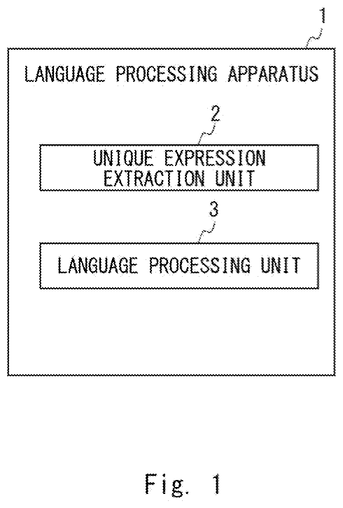

An example embodiment will be described hereinafter with reference to the drawings. FIG. 1 is a diagram for explaining a configuration of a language processing apparatus 1 according to a first example embodiment. The language processing apparatus 1 includes a unique expression extraction unit 2 and a language processing unit 3.

The unique expression extraction unit 2 extracts a unique expression related to medical care (i.e., an expression unique to medical care) from text information.

The language processing unit 3 performs language processing related to medical care based on the unique expression.

The language processing apparatus 1 extracts a unique expression from text information and performs language processing based on the unique expression. Since the unique expression is information that can be interpreted by a human, it can assist a user or the like in accounting for (i.e., explaining) the result of the language processing.

Note that the language processing apparatus 1 includes a processor, a memory, and a storage device (which are not shown in the drawing). Further, a computer program(s) in which processes in the language processing method according to this example embodiment are implemented is stored in the storage device. Further, the processor loads the computer program(s) from the storage device into the memory and executes the loaded computer program. In this way, the processor implements the functions of the unique expression extraction unit 2 and the language processing unit 3.

Alternatively, each of the unique expression extraction unit 2 and the language processing unit 3 may be implemented by dedicated hardware. Alternatively, some or all of the components of each apparatus may be implemented by general-purpose or dedicated circuitry, a processor, or a combination thereof. These components may be implemented by using a single chip or may be implemented by using a plurality of chips connected through a bus. Some or all of the components of each apparatus may be implemented by a combination of the above-described circuitry or the like and the program. Further, a CPU (Central Processing Unit), a GPU (Graphics Processing Unit), an FPGA (Field-Programmable Gate Array), or the like can be used as the processor.

### Second Example Embodiment

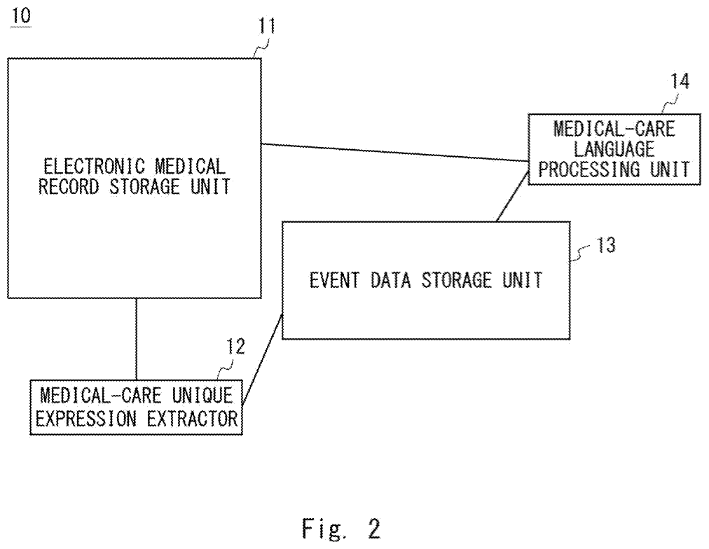

FIG. 2 is a block diagram showing a configuration of a medical language processing apparatus 10 according to a second example embodiment. The medical language processing apparatus 10 is a specific example of the language processing apparatus 1 according to the first example embodiment.

The medical language processing apparatus 10 includes an electronic medical record storage unit 11, a medical-care unique expression extractor 12, an event data storage unit 13, and a medical-care language processing unit 14.

Diagnosis/treatment records of patients are stored in the electronic medical record storage unit 11. The diagnosis/treatment records are not limited to those filled out by doctors, but represent diagnosis/treatment information in a broad sense including test records, nursing records, and the like.

If the whole information is managed by using a table, the number of blanks becomes enormous. Therefore, diagnosis/treatment records are classified into those recorded in a structured table 111 and text information 112, and are separately managed. The structured table 111 is also simply called a table.

In the structured table 111, information that can be properly managed in a table, such as attributes of patients (examples: ages, heights, weights, and genders), are recorded in the form of a table. The structured table 111 may include a plurality of tables. For example, the structured table 111 may include a table showing attributes of patients and a table showing information about diseases and treatment methods.

In the text information 112, events that occurred in patients are recorded in the form of text. The text information 112 may include subjective information, objective information, evaluation information, and planning information, all of which are recorded in SOAP-type medical records. The text information 112 is also referred to as a chronological record. The text information 112 may be expressed by vectors.

The medical-care unique expression extractor 12 is a specific example of the unique expression extraction unit 2. The medical-care unique expression extractor 12 extracts a unique expression related to medical care (called a medical-care unique expression) from the text information 112. The medical-care unique expression extractor 12 is constructed by so-called deep learning. The medical-care unique expression extractor 12 may extract a unique expression related to nursing.

For example, when the text information 112 is “There is a right-side dominant reticular shadow and tractional bronchodilation in the base of the right lung” (referred to as an input sentence 1), the medical-care unique expression extractor 12 extracts four medical-care unique expressions: “base of right lung”, “right side”, “dominant”, and “reticular shadow and traction bronchodilation”. The input sentence 1 corresponds to text information 112 that is recorded as a result of reading of radiation (e.g., X-rays).

Each of medical-care unique expressions is classified into one of predefined unique expression groups. The expressions “base of right lung” and “right side” are classified into a unique expression group representing anatomical parts. The expression “dominant” is classified into a unique expression group representing features and scales (e.g., sizes). The expression “reticular shadow and tractional bronchodilation” is classified into a unique expression group representing symptoms.

The unique expression group representing anatomical parts is expressed by and interposed between a start tag <a> and an end tag </a>. The unique expression group representing features and scales (e.g., sizes) is expressed by and interposed between a start tag <f> and an end tag </f>.

The unique expression group representing symptoms is expressed by and interposed between a start tag <d certainty={“positive” or “suspicious” or “negative” or “general” }> and an end tag </d>. The term “positive” indicates that a symptom is actually recognized; the term “suspicious” indicates that it is suspected that a patient or the like has the symptom; the term “negative” indicates that the symptom is denied; and the term “general” indicates a description of a common symptom. For example, a unique expression group representing an actually-recognized symptom is expressed by and interposed between a start tag <d certainty=“positive”> and an end tag </d>.

Therefore, the medical-care unique expression extractor 12 outputs, from the input sentence 1, an extraction result of <a>base of right lung</a><a>right side</a><f>dominant</f><d certainty=“positive”>reticular shadow and traction bronchodilation</d>. The input sentence 1 is converted into a string in which the four unique expressions are arranged (i.e., connected one to another).

Further, for example, when the text information 112 is “There is a small light-yellowish stain on wound gauze” (referred to as an input sentence 2), the medical-care unique expression extractor 12 extracts three medical-care unique expressions (e.g., unique expressions related to nursing), i.e., “wound”, “light-yellowish stain”, and “small”. The input sentence 2 corresponds to text information 112 that is recorded as a nursing record.

The term “wound” is classified into a unique expression group representing anatomical parts. The term “light-yellowish stain” is classified into a unique expression group representing symptoms. The term “small” is classified into a unique expression group representing features and scales (e.g., sizes).

Therefore, the medical-care unique expression extractor 12 outputs, from the input sentence 2, an extraction result of <a>wound</a><d certainty=“positive”>light-yellowish stain</d><f>small</f>. The input sentence 2 is converted into a string in which the three unique expressions are arranged (i.e., connected one to another).

The tags used in the above description are merely examples. Each unique expression may be expressed by and interposed between different types of tags.

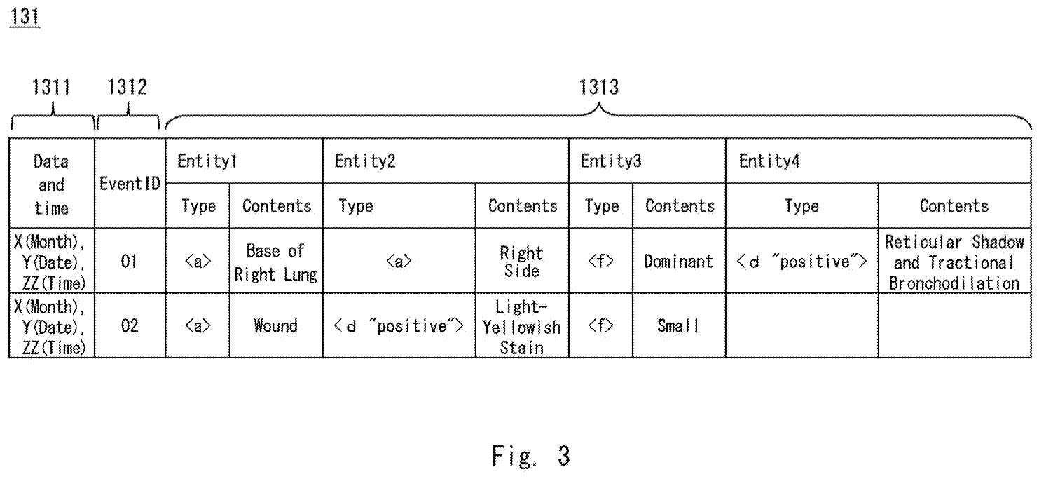

In the event data storage unit 13, the extraction result of the medical-care unique expression is stored as event data 131. FIG. 3 is a table for explaining examples of the event data 131.

The event data 131 includes date and time information 1311 about the date and time when the event occurred (e.g., X (month), Y (date), ZZ (time)), an event ID 1312 for identifying the event, and entity information 1313. The event data 131 may further include a patient ID. Further, the event data 131 may include an ID of a medical practitioner (person involved in medical care).

The entity information 1313 represents a unique expression group (also called a type) and a content (content) for each unique expression (also called an entity). FIG. 3 shows the entity information 1313 extracted from the input sentence 1 and the entity information 1313 extracted from the input sentence 2. For example, the type of the entity 1 having the content “base of right lung” is an anatomical part and represented by <a>.

Referring to FIG. 2 again, the medical-care language processing unit 14 is a specific example of the language processing unit 3. The medical-care language processing unit 14 performs medical language processing based on the structured table 111 and the event data 131. The medical-care language processing unit 14 may be an AI (Artificial Intelligence) model constructed through machine learning. The medical-care language processing unit 14 may select, for example, a unique expression related to a major event that occurred in the patient from the event data 131, and perform language processing based on the selected unique expression. The major event is an event related to target language processing (e.g., generation of a medical document). For example, when preparing a description for a surgical operation or the like, an event that contains information about a symptom of a patient is a major event.

Specifically, the medical-care language processing unit 14 generates a medical document. The medical document may be a medical text. The medical-care language processing unit 14 may generate a medical document by referring to an external database in which templates of medical documents are recorded. Note that the document generated by the medical-care language processing unit 14 may be used as a draft.

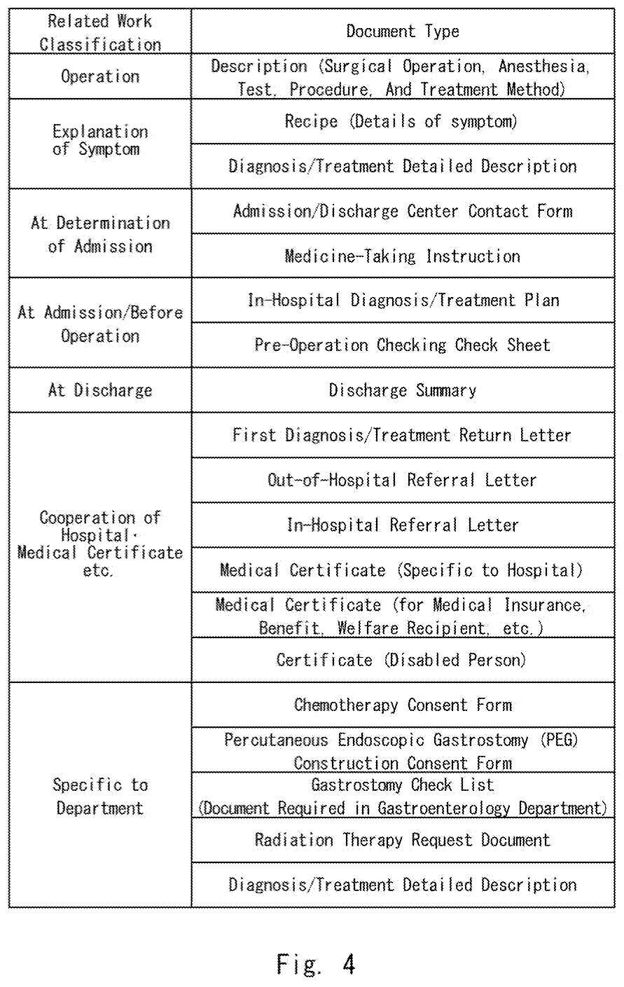

FIG. 4 is a table for explaining examples of medical documents generated by the medical-care language processing unit 14. A document to be prepared is specified for each work. A description (a surgical operation, anesthesia, a test, a procedure, and a treatment method) may be generated in relation to operation work. A recipe (details of a symptom) or a diagnosis/treatment detailed description may be generated in relation to a symptom explanation. An admission/discharge center contact form or a medicine-taking instruction request may be generated when the admission/discharge of a patient is determined. An in-hospital diagnosis/treatment plan or a pre-operation checking check sheet may be generated when a patient is admitted or before a patient undergoes a surgical operation. A discharge summary may be generated when a patient is discharged. A first diagnosis/treatment return letter, an out-of-hospital referral letter, an in-hospital referral letter, and a hospital-specific medical certificate may be generated when the hospital or the like cooperates with other medical institutions or when a patient is diagnosed. Further, a medical certificate that is used to claim a medical insurance or a benefit, a medical certificate for a welfare recipient, and a medical certificate for a disabled person may be generated. Further, the medical-care language processing unit 14 may generate a medical document specific to each medical department (example: a gastroenterology department and a radiology department). For example, a chemotherapy consent form, a Percutaneous Endoscopic Gastrostomy construction consent form, a gastrostomy checklist, a radiation therapy request document, and a diagnosis/treatment detailed description, and the like may be generated. For example, the gastrostomy checklist is a document that is required in a gastroenterology department.

For example, the discharge summary is prepared to convey information to a hospital or the like to which a patient is transferred when the patient is discharged from the hospital. The discharge summary is prepared by a doctor or a nurse. The discharge summary may include, in addition to basic information about a patient, information about the progress of a disease and a problem in nursing. In this case, the medical-care language processing unit 14 can create a document about the basic information of the patient based on the structured table 111, and can create a document about the progress of the disease or a problem in nursing based on the event data 131. The problem in nursing can be extracted, for example, from the text information 112 contained in a SOAP-type electronic medical record.

Lastly, advantageous effects provided by the second example embodiment will be described. As a method for performing medical language processing, it is conceivable to construct an AI model in which text information contained in an electronic medical record is expressed by vectors and language processing is performed based on the vectors. For example, it is conceivable to carry out the above-described method by using technologies such as context grasping, a deep language model such as BERT (Bidirectional Encoder Representations from Transformers), a reservoir, and a word vector. However, in the case of using deep learning-based AI, it is not possible to understand how language processing has been performed even when the vectors based on which the language processing has been performed and the result of the language processing (e.g., medical documents) are examined. Therefore, there has been a problem that accountability required in a medical institution cannot be fulfilled.

In the second example embodiment, event data 131 is temporarily prepared from the text information 112, which are expressed by vectors or the like, and language processing is performed based on the event data 131. The event data 131 is intermediate data that can be interpreted by humans and easily handled by machines. According to the second example embodiment, it is possible to both mechanically perform medical language processing and explain the reasons based on which the result of the medical language processing has been obtained.

Note that the present invention is not limited to the above-described example embodiments, and they can be modified as appropriate without departing from the scope and spirit of the invention.

According to the present disclosure, it is possible to provide a language processing apparatus, a language processing method, and a program capable of assisting a user or the like in accounting for the result of language processing.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line. The first and second embodiments can be combined as desirable by one of ordinary skill in the art.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

What is claimed is:

1. A language processing apparatus comprising:
at least one memory storing instructions; and
at least one processor configured to execute the instructions to:
extract a unique expression related to medical care from text information; and
perform language processing related to medical care based on the unique expression.

2. The language processing apparatus according to claim 1, wherein
information on a patient is divided into and separately managed as information recorded in a structured table and the text information,
the at least one processor is further configured to execute the instructions to perform the language processing based on the table and the unique expression.

3. The language processing apparatus according to claim 2, wherein the text information indicates an event that occurred in the patient in a medical institution.

4. The language processing apparatus according to claim 3, wherein the at least one processor is further configured to execute the instructions to select the unique expression related to the event related to the language processing, and perform the language processing based on the selected unique expression.

5. The language processing apparatus according to claim 1, wherein the at least one processor is further configured to execute the instructions to extract the unique expression from the text information expressed by a vector.

6. The language processing apparatus according to claim 1, wherein the at least one processor is further configured to execute the instructions to extract the unique expression related to nursing.

7. The language processing apparatus according to claim 1, wherein the at least one processor is further configured to execute the instructions to prepare a medical document by using a template.

8. A language processing method comprising:
extracting, by a computer, a unique expression related to medical care from text information; and
performing, by the computer, language processing related to medical care based on the unique expression.

9. A non-transitory computer readable medium storing a program for causing a computer to perform:
a process for extracting a unique expression related to medical care from text information; and
a process for performing language processing related to medical care based on the unique expression.
